(19) United States
(12) Patent Application Publication
(10) Pub. No.: US 2008/0242941 A1
(43) Pub. Date: Oct. 2, 2008

(54) Apparatus for oral examination

(76) Inventors: John Jungyoon Kim, Las Vegas, NV (US); James Jian Yu Chang, Los Angeles, CA (US)

Correspondence Address: Law Office of Michael N. Cohen, P.C., 9025 Wilshire Blvd., Suite 301, Beverly Hills, CA, 90211, US

(21) Appl. No.: 11/731,085

(22) Filed: Mar. 30, 2007

(51) Int. Cl. A61B 13/00

(52) U.S. Cl. 600/241

(57) ABSTRACT

The present invention relates to an accessory apparatus for use with conventional tongue depressors. The apparatus comprises a platform, a lens/guard piece wherein said lens/guard has a diameter larger than an average adult's open mouth, and a hinge unit for attachment of said lens/guard to said platform. The apparatus may further comprise a light source and a USB camera.

### BACKGROUND OF INVENTION

The present invention relates to a medical examination apparatus for facilitating the examination of the mouth, throat, and/or surrounding tissues of an individual. Specifically, the present invention is an accessory for attachment and use with conventional tongue depressors.

Despite advances in medical technology, health professionals routinely use low-tech, conventional tongue depressors to examine a patient's mouth, throat, and surrounding tissues. Conventional tongue depressors are disposable, flat wooden or plastic instruments that are inserted in an individual's mouth and used to minimize the obstruction an individual's tongue poses in examining his mouth and throat. Tongue depressors also may be used to elicit a gag reflex, which may provide an enhanced view of a patient's throat.

Shortcomings of conventional tongue depressors used alone are numerous. First, a separate light source is required to adequately view a patient's throat; second, because tongue depressors may trigger a patient's gag reflex, a health care professional is not protected from saliva or vomit that may be expelled from the patient; third, a separate magnifying device may be required to adequately view areas of a patient's mouth and/or throat; and fourth, in examining the throat of a patient, certain views of a patient's throat may be transient and a health professional cannot capture views in a fixed medium. Improvements over conventional tongue depressors have addressed some of the above shortcomings.

U.S. Pat. No. 4,697,578 (hereinafter referred to as the '578 patent) teaches an apparatus that is used to examine a person's mouth and throat that includes a light source and viewing lens. Shortcomings of the apparatus taught by '578 patent include the inability to conveniently hold the apparatus in a coat pocket. The '578 device is bulky and does not include means for conversion into a more compact size.

In addition, a non-disposable tongue depressor is incorporated in the apparatus and must be sterilized for use with each patient. This may be impractical for health professionals with busy practices and numerous patients to treat in a day. Also, no means are provided to protect the examining health professional from being inadvertently struck by a patient's saliva or vomit.

Furthermore, no means is provided by the '578 patent to capture views of the patient's mouth and/or throat in a fixed medium such as film or digital format.

U.S. Pat. No. 5,656,014 (hereinafter referred to as the '014 patent) teaches a modified tongue depressor that comprises a light source, and a curved elongated member for depressing a patient's tongue. The '014 apparatus does not address the aforementioned shortcomings of the prior art. Specifically, the '014 patent does not provide an enhanced viewing lens, nor does it provide protection from a patient's saliva or vomit. Neither does it comprise means to capture images in a fixed medium.

Furthermore, because the tongue depressor component of the '014 patent is modified and non-disposable, the '014 patent requires the user to employ sterile covers especially designed for said apparatus. This forces the health professional to purchase such specialized, sterile covers, since the tongue depressor component of the '014 patent has a non-conventional configuration.

U.S. Pat. No. 6,350,235 (hereinafter referred to as '235 patent) teaches an apparatus that incorporates a unit having the ability for attachment to conventional tongue depressors. Said unit consists of a light source, as well as means to capture views of a patient's mouth, and convey the images to an external computer monitor station. The '235 patent does not provide for a guard from saliva or vomit. Such a guard would be particularly necessary in said device for protection of expensive components such as the image capturing means. Nor does the '235 apparatus provide means to magnify the view of the patient's mouth for the examining health professional. Additionally, practice of the '235 invention requires a relatively complicated IT arrangement, as computer monitor stations are incorporated for use.

In view of the above shortcomings of the above improvements over the prior art, conventional tongue depressors continue to be the most commonly and widely used tool for examination of an individual's mouth, throat, and surrounding tissue. The prior art improvements fail to address all of the shortcomings of conventional tongue depressors and do not provide a low-tech, compact, and convenient apparatus to improve examination with wooden, disposable tongue depressors alone.

Therefore, it is an object of the present invention to address all of the above shortcomings, in a manner that is simple for the examining health professional to practice.

It is also an object of the present invention to teach an apparatus that aids in the examination of an individual's mouth, throat, and surrounding tissue.

It is another object of the present invention to teach a medical apparatus that is convenient to store in the pocket of the user.

It is an additional object of the present invention to teach a medical apparatus for oral examination that protects the examiner from contact with saliva or vomit from a patient being examined.

### SUMMARY OF INVENTION

The present invention relates to an accessory for improving the examination of an individual's mouth, throat, and surrounding tissue. The present invention incorporates the use of a conventional tongue depressor, typically constructed from disposable wood or plastic. A viewing lens, having dimensions larger than an average person's mouth, serves two purposes: 1) first, it acts as a guard to protect the examining health professional from contact with a patient's saliva and/or vomit; 2) the viewing lens also serves to magnify the mouth and throat of the patient being examined. A light source is also provided as well as housing to enclose a power source for the light. A means of taking digital images is incorporated within the lens/guard to allow a health professional to capture views of a patient's mouth and throat within a fixed medium.

### BRIEF DESCRIPTION OF THE DRAWINGS

FIG. 1 shows a perspective view of the present invention with a lens/guard placed in a compact position.

FIG. 2 shows a perspective view of the present invention with a lens/guard placed in an open position.

FIG. 3 shows a view of the present invention in use on a patient.

### DETAILED DESCRIPTION OF THE PRESENT INVENTION

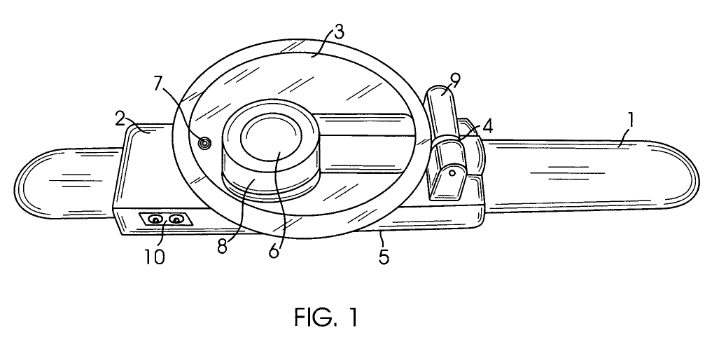

FIG. 1 shows a perspective view of the present invention in conjunction with a conventional tongue depressor. The present invention is an accessory apparatus for use with a conventional tongue depressor 1 comprising a platform 2, a viewing lens/guard 3, a light source 4, a means to power the light source 5, a switch for turning on and off the light source 6, a USB camera 7, and a button for powering the camera 8.

The lens/guard is attached to the platform through a hinged unit 9, allowing the lens/guard to pivot from an open position to a closed position. The hinged unit allows for 180 degree movement of the lens/guard.

FIG. 1 depicts the apparatus in a closed, compact position. In the closed position, the apparatus and tongue depressor assembly may easily fit in the coat pocket of a health professional.

A switch 6 for powering the light source on and off is located within the platform 2. It is positioned on the end of the platform that is proximal to a health professional examining a patient. The switch may be configured to have a shape and dimension convenient for the examining health professional to power the switch with his thumb or index finger. In the preferred embodiment of the present invention the switch has a circular configuration approximating the area of a person's thumb.

The platform houses means to power the light source. In the preferred embodiment of the present invention an LED light source is used and a suitable battery, known in the art, is used to power the LED light source. By employing an LED light source, low power consumption is used, allowing for infrequent battery replacement.

The present invention may further comprise a USB camera 7 known in the art. The camera may be positioned within the lens/guard. A button 8 is provided within the platform for recording an image. In the preferred embodiment of the present invention, the button for recording images is incorporated beneath and adjacent to the light switch so that when the user fully depresses the switch, both the light is powered on and a picture is taken. The platform houses a connection means 10 for a USB outlet. A user can attach a USB cable to a printer or personal computer and download recorded images of a patient's mouth.

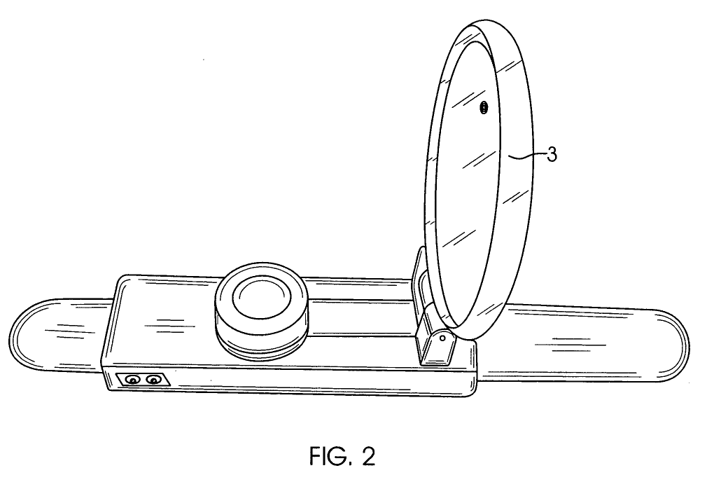

FIG. 2 shows the apparatus with the lens/guard 3 pivoted in an open position. The lens/guard should have a diameter that is larger than an average person's fully opened mouth. The lens/guard serves two purposes: first the size of the lens/guard protects an examining health professional from direct contact with saliva or vomit of a patient. Because the nature of an examination with a tongue depressor may elicit a gag reflex in a patient, a patient may vomit or expel saliva while being examined. The lens guard minimizes exposure a health professional has with a patient's saliva or vomit, reducing transmission of infections to the health professional as well as other patients.

Furthermore, the lens/guard is constructed from a single, convex lens that allows for magnification of images beneath it. Thus the lens allows for views of the patient's mouth and throat to be enlarged, improving the health professional's ability to examine the patient.

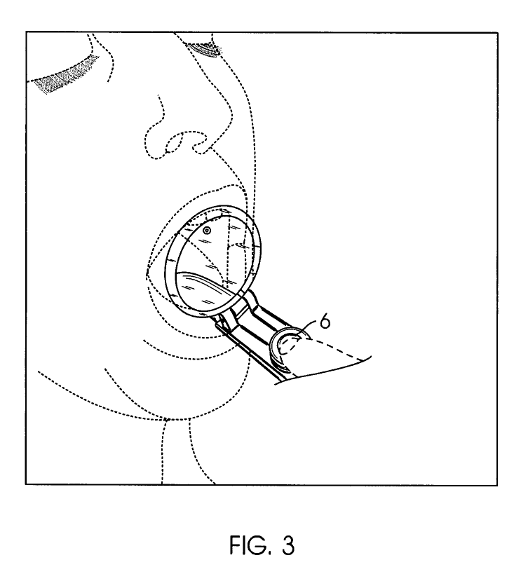

FIG. 3 depicts the present invention in use on a patient. By slightly depressing the light source switch 6, a patient's mouth is illuminated. Fully depressing the light source button triggers the USB camera button, allowing for the recording of an image. As seen in FIG. 3, the lens/guard obstructs the opening of the patient's mouth, and minimizes airborne transmission of infections from the patient.

With respect to the above description, it is to be realized that the optimum dimensional relationships for the parts of the invention, to include variations in size, materials, shape, form, function, manner and use are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A tongue depressor accessory for use with conventional tongue depressors comprising:
A platform;
A lens/guard piece wherein said lens/guard has a diameter larger than an average adult's open mouth; and
A hinge unit for attachment of said lens/guard to said platform.

2. The tongue depressor accessory of claim 1 further comprising a light source and energy source of powering said light source.

3. The tongue depressor of claim 1 further comprising a USB camera and means for recording images with said camera.
